(19) United States
(12) Patent Application Publication
(10) Pub. No.: US 2024/0207650 A1
(43) Pub. Date: Jun. 27, 2024

(54) ULTRASOUND-INDUCED MICROBUBBLE DRUG DELIVERY SYSTEM LOADING STABILIZED HYDROPHOBIC DRUG WITH POLYETHYLENE GLYCOL BASED NONIONIC SURFACTANT AND METHOD OF MANUFACTURING THE SAME

(71) Applicants: SAHMYOOK UNIVERSITY INDUSTRY-ACADEMIC COOPERATION FOUNDATION, Seoul (KR); N TO B CO., LTD., Seoul (KR)

(72) Inventors: Myoung Hwan PARK, Guri-si (KR); Jung Min LEE, Guri-si (KR); Been YOON, Seoul (KR)

(73) Assignees: SAHMYOOK UNIVERSITY INDUSTRY-ACADEMIC COOPERATION FOUNDATION, Seoul (KR); N TO B CO., LTD., Seoul (KR)

(21) Appl. No.: 18/230,747

(22) Filed: Aug. 7, 2023

(30) Foreign Application Priority Data
Dec. 27, 2022 (KR) 10-2022-0185355

(51) Int. Cl. A61N 7/00

(52) U.S. Cl. CPC A61N 7/00; A61N 2007/0039

(57) ABSTRACT

Provided is a method of manufacturing a drug delivery system including a first surfactant and a second surfactant, the drug delivery system manufactured by using the method, and a contrast medium, wherein the first surfactant includes a polyethylene glycol based nonionic surfactant and the second surfactant includes a Tween-type surfactant or a SPAN-type surfactant. According to the present invention, the microbubble including the first surfactant, which includes a polyethylene glycol (PEG) based nonionic surfactant, and the second surfactant, which includes a Tween-type surfactant or a SPAN-type surfactant, may stably have uniform size distribution, may include high loading amount of drugs and dye on the surface of the delivery system, and thereby, may be efficiently used as a drug delivery system. Also, drug release may increase by ultrasonic wave, which is external stimulus, from the microbubble and thereby, more efficient drug delivery may be available.

### CROSS-REFERENCE TO RELATED PATENT APPLICATION

This application claims the benefit of Korean Patent Application No. 10-2022-0185355, filed on 27 Dec. 2022, in the Korean Intellectual Property Office, the disclosure of which is incorporated herein in its entirety by reference.

### BACKGROUND OF THE INVENTION

### 1. Field of the Invention

The present invention relates to an ultrasound-induced microbubble drug delivery system loading a stabilized hydrophobic drug with polyethylene glycol based nonionic surfactant and a method of manufacturing the same, and more particularly, to a method of manufacturing a drug delivery system including a first surfactant and a second surfactant, the drug delivery system manufactured by using the method, and a contrast medium, wherein the first surfactant includes a polyethylene glycol based nonionic surfactant and the second surfactant includes a Tween-type surfactant or a SPAN-type surfactant.

### 2. Description of the Related Art

A drug delivery system is used to selectively deliver drugs to targeted parts and to optimize effective blood concentration for a long period of time according to types of diseases so that the effects and benefits of medical treatment may be improved and adverse drug reactions may be minimized.

In particular, a drug delivery platform which may effectively release and control drugs in response to external stimulus such as electricity, light, and ultrasonic waves has developed along with various medical equipment. Accordingly, drug injection may be effectively programmed so as to optimize patient care. The ultrasonic waves is the most harmless medical equipment from among such external stimulus, and microbubble, which is used as an ultrasound contrast agent and is formed of biomaterials including a small quantity of gas and phosphatide, may be used as an excellent drug delivery system.

Such a drug delivery system may be manufactured by using various methods. One of the representative methods includes use of a liposome based microbubble drug delivery system formed of phosphatide or protein, a polymer based microbubble drug delivery system manufactured by using dual emulsion solvent evaporation, and a Micelle microbubble drug delivery system using α-tocopherol derivatives.

In order to facilitate drug delivery efficiency in the drug delivery system, researches on maximization of drug delivery have been made in various areas in such a way that various target ligands are combined with particles of the delivery system improve target delivery efficiency, drug release speed is controlled through near-infrared irradiation, or penetration capability is improved by ultrasonic waves. However, there is still a demand for the development of an excellent drug delivery technique that may control appropriate time and a proper amount of drug release to target parts.

In a general drug delivery system, when a shell has a small thickness, a loading amount of drugs is low and when a shall is hard, drugs are not released due to external stimulus or drug agglomerate due to a polymer shell. Accordingly, there is a need for the development of a drug delivery system that may overcome existing problems.

### SUMMARY OF THE INVENTION

The present invention provides a method of manufacturing a microbubble drug delivery system.

The present invention also provides the microbubble drug delivery system manufactured by using the above method and a contrast medium.

Unless defined differently, all terms used in the description of the invention have the same meaning as generally understood by those skilled in the art. Also, it should be understood, however, that preferred methods or samples are illustrated in the description, their similarities or equivalents are also included in the scope of the invention. In addition, although not described, figures illustrated in the description are intended to include the meaning “about”. It will be further understood that if a part “includes” an element, it does not denote exclusion of other elements and instead, further inclusion of another elements, unless defined contrarily. All publications stated as references of the present invention are integrated as references of the description.

According to an aspect of the present invention, there is provided a method of manufacturing an ultrasound-induced microbubble drug delivery system comprising: (a) manufacturing a mixture by dissolving drugs, dye, or a combination thereof; a first surfactant comprising a polyethylene glycol (PEG) based nonionic surfactant; and a second surfactant comprising a Tween-type surfactant or a SPAN-type surfactant, in an organic solvent; (b) manufacturing a concentrate by removing an organic solvent from the mixture; and (c) manufacturing microbubble using a sonicator after putting liquid gas into the manufactured concentrate.

The dye may include at least one selected from the group consisting of Cy3, Cy3.5, Cy5, Cy5.5, Cy7, Cy7.5, sulfo-Cy3, sulfo-Cy5, sulfo-Cy7, IR140, IR780, IR820, Flamma® 552 Vinylsulfone, Flamma® 581 Vinylsulfone, Flamma® 594 Vinylsulfone, Flamma® 648 Vinylsulfone, Flamma® 749 Vinylsulfone, Flamma® 774 Vinylsulfone, Alexa Fluor® 555, Alexa Fluor® 594, Alexa Fluor® 680, Alexa Fluor® 750, CF770, IRDye 680LT, IRDye 750, DyLight 549, DyLight 594, DyLight 650, DyLight 680, DyLight 755, ATTO 550, and ATTO 647N.

The organic solvent may include dichloromethane, ethyl acetate, acetone, ethanol, methanol, methyl ethyl ketone, methylene chloride, dichloroethane, chloroform, dioxane, dimethyl sulfoxide, acetonitrile, acetic acid, or combinations thereof.

The microbubble manufactured by using the method above may have a size of about 0.5 μm through 30 μm, about 0.5 μm through 20 μm, about 0.5 μm through 15 μm, about 0.5 μm through 10 μm, about 1 μm through 30 μm, about 1 μm through 20 μm, about 1 μm through 15 μm, about 1 μm through 10 μm, about 1 μm through 5 μm, or about 1 μm through 4 μm.

The drug delivery system may be dispersed in an emulsion-form aqueous solution.

The liquid gas may include Perfluoro-n-pentane, 1H-Undecafluoropentane, or 2H,3H-perfluoropentane. As the drug delivery system according to an embodiment of the present invention includes the liquid gas in inner core thereof, stability may be higher than a microbubble including general gas.

The polyethylene glycol (PEG) based nonionic surfactant may be selected from the group consisting of Pluronic F-127, polyethylene oxide-polypropylene oxide-polyethylene oxide (PEO-PPO-PEO), polystyrene-co-maleic anhydride, polyethylene glycol-poly L-lactic acid-polyethylene glycol (PEG-PLLA-PEG), a copolymer of polyethylene oxide and polypropylene oxide, and combinations thereof.

In the microbubble drug delivery system according to an embodiment of the present invention, a loading amount of drugs may increase due to a characteristic of gel of the polyethylene glycol (PEG) based nonionic surfactant. Also, the drug delivery system may be well mixed with loaded drugs and thereby, may be used as a stable drug delivery system without drug agglomerate. In addition, the drug delivery system may not be granulated and thereby, have high sensitivity to external stimulus (for example, ultrasonic wave). Accordingly, drug release may increase.

The drugs may be fat soluble drugs (hydrophobic drugs) and may include all drugs having affinity with geological features, lipoid, and nonpolar fluid. Also, kinds of drugs are not particularly restricted and may all include chemotherapy drugs, protein drugs, peptide drug, nucleic acid molecules for gene therapy, nanoparticle, iodized contrast medium, gadolinium contrast medium, barium contrast medium, sulfa hexafluoride, contrast substances such as fluorescent and magnetic particles, (functional) cosmetic active ingredient, or cosmetologically used active ingredient. For example, the cosmetic active ingredient or the cosmetologically used active ingredient may include niacinamide, arbutin, Selina, 4-n-butylresorcinol, ethyl ascorbyl ether having whitening effect; adenosine, asiaticoside, retinol, retinyl palmitate having anti-wrinkle effect; allantoin, aloe vera extracts, azulene, Asiatic pennywort extract having anti-inflammatory effect; and other cosmetic ingredients having antioxidant effect or UV blocking effect, however, is not limited thereto.

Also, the fat soluble drugs may include, for example, anticancer drugs, (degenerative) brain diseases drugs, anti-inflammatory drugs, pain relievers, antarthritic, antispasmodic drugs, antidepressants, antipsychotic drugs, tranquilizer, antianxiety drugs, narcotic antagonists, anti-Parkinson drugs, cholinergic agonist, anti-angiogenic inhibitors, immunosuppressants, antiviral agents, antibiotics, anorectic agents, anticholinergic drugs, antihistaminic agents, antimigraine drugs, hormone drugs, coronary, cerebrovascular, or peripheral vascular vasodilator, contraceptive pills, antithrombotic, diuretics, antihypertensive drugs, drugs for cardiovascular diseases, and cosmetic ingredients (for example, anti-wrinkle agents, skin-aging suppressants, and skin whitening agents), however, are not limited thereto. As an example, the drugs may be doxorubicin, curcumin, Paclitaxel, vincristine, daunorubicin, vinblastine, actinomycin-D, doxetaxel, etoposide, teniposide, bisantrene, homoharringtonine, Gleevec; STI-571, cisplatin, 5-fluorouracil, Adriamycin, methotrexate, busulfan, chlorambucil, cyclophosphamide, melphalan, nitrogen mustard, or nitrosourea.

The Tween-type surfactant may be at least one selected from the group consisting of Tween® 20 (Polyoxyethylene (20) sorbitan monolaurate), Tween® 40 (Polyoxyethylene (20) sorbitan monopalmitate), Tween® 60 (Polyoxyethylene (20) sorbitan monostearate), and Tween®80 (Polyoxyethylene (20) sorbitan monooleate), however, is not limited thereto. The SPAN-type surfactant may be at least one selected from the group consisting of sorbitan laurate, sorbitan palmirate, sorbitan stearate, and sorbitan oleate, however, is not limited thereto.

The drugs may be dissolved in an organic solvent with the concentration of 0.001 through 10 g/mL. Also, organic solvent above has a low boiling point within a solution where the drugs are dissolved and needs to be completely removed by using a vacuum evaporator or a rotary evaporator.

The microbubble drug delivery system may include the first surfactant and the second surfactant in weight ratio of 1:0 through 1:1, and more preferably, 1:1 through 20:1, 4:1 through 20:1, or 5:1 through 20:1.

According to another aspect of the present invention, there is provided a microbubble drug delivery system manufactured by using the method described above and a contrast medium.

When, in particular, ultrasonic waves is applied to the drug delivery system, the microbubble is broken or agglomerated and high temperature and high pressure instantaneously occur. In this regard, drug delivery efficiency may be maximized and thereby, drug release may be increased. Such ultrasonic waves is not particularly restricted and for example, may be focused ultrasound. In particular, the focused ultrasound may be used to efficiently control the drug release of a selected part of a body from the outside.

The drug delivery system may include fat soluble drugs. For example, the fat soluble drugs may include doxorubicin, curcumin, Paclitaxel, vincristine, daunorubicin, vinblastine, actinomycin-D, doxetaxel, etoposide, teniposide, bisantrene, homoharringtonine, Gleevec; STI-571, cisplatin, 5-fluorouracil, Adriamycin, methotrexate, busulfan, chlorambucil, cyclophosphamide, melphalan, nitrogen mustard, or nitrosourea.

When, in particular, ultrasonic waves is applied to the drug delivery system, the microbubble is broken or agglomerated and high temperature and high pressure instantaneously occur. In this regard, drug delivery efficiency may be maximized and thereby, drug release may be increased. Such ultrasonic waves is not particularly restricted and for example, may be focused ultrasound. In particular, the focused ultrasound may be used to efficiently control the drug release of a selected part of a body from the outside. The frequency of the ultrasound which maximizes the drug delivery efficiency may be 1 MHz through 50 MHZ, 1 MHz through 30 MHZ, 1 MHz through 20 MHz, or 1 MHz through 10 MHz. The intensity of the ultrasound which maximizes the drug delivery efficiency may be about, for example, 1 W through 100 W, 1 W through 80 W, 1 W through 70 W, 1 W through 50 W, 1 W through 40 W, 1 W through 30 W, 1 W through 20 W, or 1 W through 15 W.

The term “contrast medium” denotes a medicine used to closely view blood vessels or tissues by artificially making a contrast difference and showing the difference into an image, in order to diagnose conditions of body organs and diseases, and more specifically, may denote an “ultrasound contrast medium.” A representative example of the ultrasound contrast medium may be fine bubble or microbubble. Here, a contrast characteristic may be indicated due to a difference in ultrasonic reactivity generated from the surface of fine bubble or microbubble entered into a body.

The drug delivery system using a plurality of fine bubble or microbubble including dyes is also used as a contrast medium. Also, as the drug delivery system manufactured by using the method described above may simultaneously perform diagnosis and care, biocompatibility and in vivo stability may be excellent and microbubbles may be reacted along with ultrasonic waves. Accordingly, the drug delivery system may be efficiently used as an ultrasound contrast medium.

In addition, according to diagnosis and purpose of tests, when iodized contrast medium, gadolinium contrast medium, barium contrast medium, and contrast substances such as fluorescent and magnetic particles, which are well-known in the art, are used in the microbubble, the microbubble including such materials may be used as contrast medium having various characteristics. The contrast medium illustrated above may be applied to X-ray imaging technique, Computer Tomography (CT), Magnetic Resonance Imaging (MRI), Positron Emission Tomography (PET), and nuclear imaging including ultrasonography, however, is not particularly restricted thereto.

According to another aspect of the present invention, there is provided a pharmaceutical composition for cancer treatment including the microbubble drug delivery system manufactured by using the method described above.

The term “cancer” used herein denotes diseases generated by cells having an aggressive characteristic, in which cells are divided and grown by ignoring its normal growth limit, an invasive characteristic, in which cells invade neighboring tissues, and a metastatic characteristic, in which cells spread to other parts of a body. In the present invention, the cancer may have the same meaning as a malignant tumor.

The cancer may be, for example, any one selected from the group consisting of esophageal cancer, breast cancer, thyroid cancer, bladder cancer, liver cancer, encephaloma, gastric cancer, cholangiocarcinoma, pancreatic cancer, colon cancer, lung cancer, thymic carcinoma, mesothelioma, ovarian cancer, endometrial cancer, cervical cancer, uterine serous carcinoma (USC), non-small cell lung cancer, and acute myeloid leukemia (AML), however, is not limited thereto.

When the composition including the microbubble drug delivery system is used as the pharmaceutical composition, the drug delivery system may include the active components of about 0.0001 through 50 weight % with respect to the total weight of the composition.

The composition may additionally contain one or more active components having the same or similar functions with the active components above.

The composition may include one or more pharmaceutically allowable carriers to be administered, in addition to the active components illustrated above. The pharmaceutically allowable carrier may be saline, sterilized water, Ringer's solution, buffered saline, dextrose solution, maltodextrin solution, glycerol, ethanol, liposome, and a mixture of at least one carrier above. If needed, other general additives such as antioxidant, buffer solution, and bacteriostatic agents may be also added. Also, diluent, dispersants, surfactants, and lubricants may be further added to pharmaceutically prepare injectable formulations such as aqueous solutions, suspensions, and emulsions, pills, capsules, granules, or tablets. In addition, in order to specifically function to a target organ, a target organ-specific antibody or other ligand may be combined with a carrier. Furthermore, pharmaceutical preparation may be available according to each disease or component by using an appropriate method well-known in the art or a method disclosed in Remington's literature.

The drug delivery system may be administered through routes such as intravenous, intraperitoneal, intramuscular, intrathecal, intracerebroventricular, subcutaneous, intradermal, nasal, mucosal, inhalation, and oral so as to be delivered into the body. A dosage may vary according to recipient's weight, age, gender, physical condition, diet, administration time, administration methods, excretion rate, and severity of disease.

According to another aspect of the present invention, there is provided a method of delivering drugs comprising: administering a drug delivery system including microbubbles loading drugs or dyes to an individual; and irradiating ultrasonic waves to parts to which the drug delivery system is administered and releasing drugs. The drugs are described above.

The drug delivery system may be administered through routes such as intravenous, intraperitoneal, intramuscular, intrathecal, intracerebroventricular, subcutaneous, intradermal, nasal, mucosal, inhalation, and oral so as to be delivered into the body.

When, in particular, ultrasonic waves is applied to the drug delivery system, the microbubble is broken or agglomerated and high temperature and high pressure instantaneously occur. In this regard, drug delivery efficiency may be maximized. More specifically, such ultrasonic waves may be focused ultrasound, however, is not particularly restricted thereto. For example, the method of delivering drugs according to the present invention uses blood-brain barrier (BBB) disruption induced by ultrasonic irradiation. When the drug delivery system is administered to an individual and ultrasonic waves is irradiated to administered parts, the BBB disruption instantaneously occurs due to hyperthermia from the ultrasonic waves and drug permeability into brain parenchyma may be increased.

The individual may be mammals including mice, rats, dogs, cats, cows, horses, pigs, and human, however, is not particularly restricted thereto. The administration of the drug delivery system may be performed by appropriately selecting a method well-known in the art in consideration of kinds of disease, affected parts, and conditions.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates views of microbubbles including first and second surfactants each of which carries the surfactants only, dye, drugs, and both dye and drugs identified by an optical microscope;

FIG. 2 respectively illustrates microbubbles including first and second surfactants each of which carries doxorubicin DOX@MB and curcumin Curcumin@MB as drugs and IR140 IR140@MB as dye identified by a confocal microscope;

FIG. 3 is a graph showing a result of size distribution of microbubbles including first and second surfactants each of which carries the surfactants only, dye, drugs, and both dye and drugs identified by a dynamic light scattering (DLS) particle size analyzer;

FIGS. 4A and 4B are graphs showing results of drug release experiments of a manufactured microbubble drug delivery system by using ultrasonic irradiation each of which is a curcumin release experiment and a paclitaxel release experiment;

FIG. 5 is a graph showing cytotoxicity of microbubble including first and second surfactants MB and the microbubble loading paclitaxel PTX@MB; and

FIG. 6 shows an absorption degree of a microbubble including first and second surfactants MB and the microbubble loading paclitaxel PTX@MB or IR140 IR140@MB as dye within a cell identified by a confocal microscope.

### DETAILED DESCRIPTION OF THE INVENTION

Hereinafter, one or more embodiments of the present invention will be described with reference to examples. However, the examples are merely representative for purposes of describing one or more embodiments and the present invention should not be construed as limited to only the examples set forth herein.

### Example 1: Manufacture of Microbubble Drug Delivery System

### Example 1-1: Manufacture of Microbubble Drug Delivery System Loading Both Drugs and Dye

2-10 mg of model drug (Paclitaxel), 50-200 mg of a first surfactant (Pluronic F-127), and 10-50 mg of a second surfactant (Tween 80) are respectively dissolved in 1 ml of Chloroform. 2-5 mg of IR140 as fluorescent dye is dissolved in 1-3 ml of Ethanol. The dissolved solution is mixed in 30 ml of a vial and then, dispersed and dissolved. An organic solvent is removed from the vial containing the mixed solution by using a rotary evaporator and then, a concentrate where the model drug, the fluorescent dye, and the surfactants are mixed is manufactured. 10-20 ml of a sodium chloride aqueous solution (0.9%) and 0.2-1 ml of 2H,3H-perfluoropentane, which is liquid gas, are put into the vial containing the concentrate and a tip sonicator is applied to the vial for 0.5-2 minutes to manufacture a microbubble drug delivery system. Centrifugation is used in the aqueous solution to remove an aqueous solution at the upper part and a precipitated microbubble drug delivery system is collected.

### Example 1-2: Manufacture of Microbubble Drug Delivery System Loading Drugs

2-10 mg of model drug (Paclitaxel), 50-200 mg of a first surfactant (Pluronic F-127), and 10-50 mg of a second surfactant (Tween 80) are respectively dissolved in 1 ml of Chloroform. The dissolved solution is mixed in 30 ml of a vial and then, dispersed and dissolved. An organic solvent is removed from the vial containing the mixed solution by using a rotary evaporator and then, a concentrate where the model drug and the surfactants are mixed is manufactured. 10-20 ml of a sodium chloride aqueous solution (0.9%) and 0.2-1 ml of 2H,3H-perfluoropentane, which is liquid gas, are put into the vial containing the concentrate and a tip sonicator is applied to the vial for 0.5-2 minutes to manufacture a microbubble drug delivery system. Centrifugation is used in the aqueous solution to remove an aqueous solution at the upper part and a precipitated microbubble drug delivery system is collected.

### Example 1-3: Manufacture of Microbubble Drug Delivery System Loading Dye

50-200 mg of a first surfactant (Pluronic F-127) and 10-50 mg of a second surfactant (Tween 80) are respectively dissolved in 1 ml of Chloroform. 2-5 mg of IR140 as fluorescent dye is dissolved in 1-3 ml of Ethanol. The dissolved solution is mixed in 30 ml of a vial and then, dispersed and dissolved. An organic solvent is removed from the vial containing the mixed solution by using a rotary evaporator and then, a concentrate where the fluorescent dye and the surfactants are mixed is manufactured. 10-20 ml of a sodium chloride aqueous solution (0.9%) and 0.2-1 ml of 2H,3H-perfluoropentane, which is liquid gas, are put into the vial containing the concentrate and a tip sonicator is applied to the vial for 0.5-2 minutes to manufacture a microbubble drug delivery system. Centrifugation is used in the aqueous solution to remove an aqueous solution at the upper part and a precipitated microbubble drug delivery system is collected.

### Example 1-4: Manufacture of Microbubble without Loading Drugs or Dye

50-200 mg of a first surfactant (Pluronic F-127) and 10-50 mg of a second surfactant (Tween 80) are respectively dissolved in 1 ml of Chloroform. The dissolved solution is mixed in 30 ml of a vial and then, dispersed and dissolved. An organic solvent is removed from the vial containing the mixed solution by using a rotary evaporator and then, a concentrate is manufactured. 10-20 ml of a sodium chloride aqueous solution (0.9%) and 0.2-1 ml of 2H,3H-perfluoropentane, which is liquid gas, are put into the vial containing the concentrate and a tip sonicator is applied to the vial for 0.5-2 minutes to manufacture a microbubble drug delivery system. Centrifugation is used in the aqueous solution to remove an aqueous solution at the upper part and a precipitated microbubble drug delivery system is collected.

Hereinafter, experiments are performed in Examples below by using composites including the drug delivery system manufactured in Examples 1-1 through 1-3.

### Example 2: Identification of Manufactured Microbubble Drug Delivery System

### Example 2-1: Size Analysis of Manufactured Microbubble Drug Delivery System by Using Optical Microscope

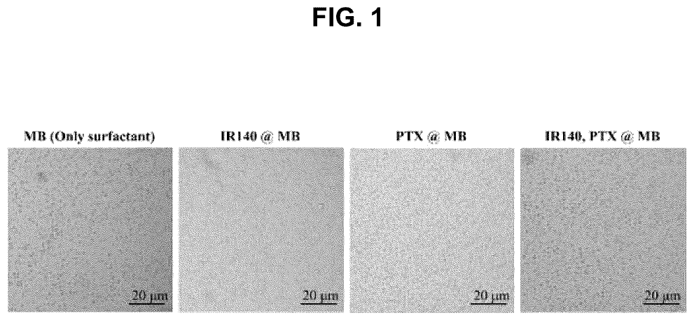

An experiment is performed to identify the microbubble drug delivery system manufactured in Example 1 by using an optical microscope OM. The microbubble MB manufactured by using the method in Example 1-4 which does not load drugs or dye, the microbubble IR140@MB manufactured by using the method in Example 1-3 which carries the dye, the microbubble PTX@MB manufactured by using the method in Example 1-2 which carries the drugs, and the microbubble IR140, PTX @MB manufactured by using the method in Example 1-1 which carries both drugs and dye are identified by an optical microscope OM and the results are shown in FIG. 1.

As identified in FIG. 1, the microbubbles MB, IR140@MB, PTX@MB, and IR140, PTX@MB including the first and second surfactants are all in the same size and are uniform. Also, the sizes thereof are 1-4 μm and thereby, structural stability is high.

### Example 2-2: Identification of Structure of Manufactured Microbubble Drug Delivery System by Using Confocal Microscope

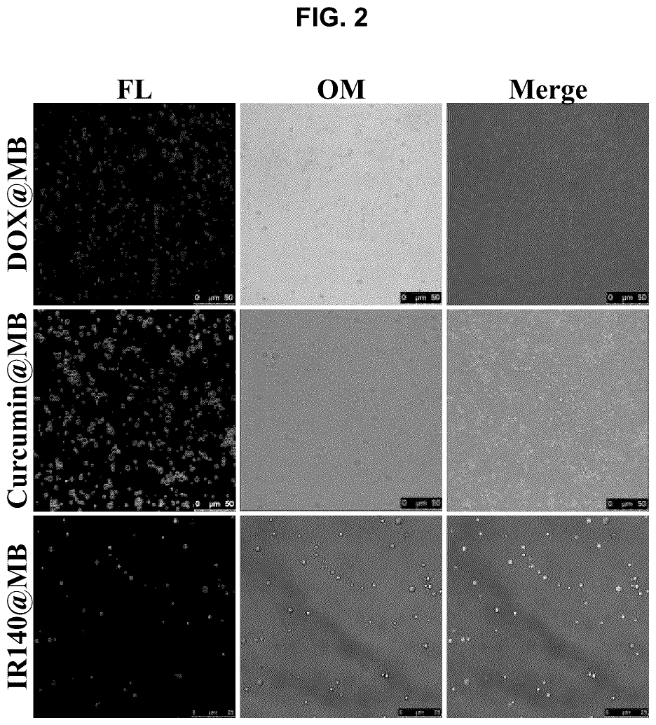

An experiment is performed to identify the manufactured microbubble drug delivery system having structural stability by using a confocal microscope, in order to visualize a structure of microbubbles loading drugs or fluorescent dye. In order to use drugs having fluorescence, doxorubicin and curcumin are used as the drugs in the microbubbles loading the drug, and the experimental method and condition are the same as those of in Example 1-2. The microbubble loading the dye used herein is the same as the microbubble manufactured by using the method in Example 1-3. The form of the manufactured microbubble drug delivery system loading the drug or the fluorescent dye is identified and the results are shown in FIG. 2.

As identified in FIG. 2, the carried drugs and dye are indicated on the surface of the microbubble. In this regard, it is concretely identified that the drugs and the dye are carried on the surfactant, which is the surface of the delivery system, in the manufactured drug delivery system. Accordingly, the drugs or the dye may be efficiently captured by critical micell concentration of the surfactants each of which is included in the manufactured microbubble.

### Example 2-3: Identification of Size Distribution of Manufactured Microbubble Drug Delivery System by Using Dynamic Light Scattering (DLS) Particle Size Analyzer

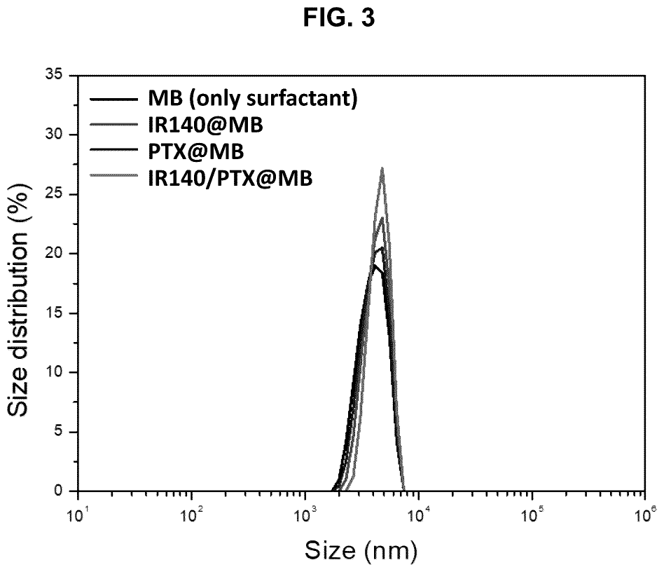

A dynamic light scattering (DLS) particle size analyzer is used to concretely identify the size distribution of the manufactured microbubble drug delivery system in which a visual size thereof is identified through the Example above. The size distribution of the microbubbles in the Examples 1-1, 1-2, 1-3, and 1-4 is respectively identified in an emulsion-formed composite by using the DLS analyzer and the results are shown in FIG. 3.

As identified in FIG. 3, all manufactured microbubbles have the similar size distribution. Accordingly, if any of the drugs and dyes are carried, the microbubbles having uniform sizes may be manufactured and thereby, structural stability may be high.

In this regard, the manufactured microbubbles may have the small size of 1-4 μm and thereby, structural stability may be high.

### Example 3: Drug Release Experiment of Manufactured Microbubble Drug Delivery System by Using Ultrasonic Irradiation

An experiment is performed to identify how fast the drugs may release by focused ultrasound, which is external stimulus, in the microbubbles loading the drugs.

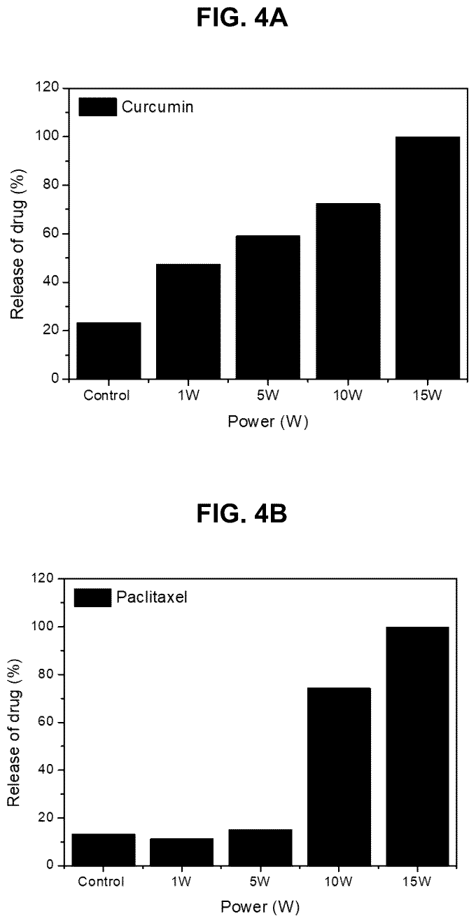

The drug delivery system dispersed in 30 ml of vial is manufactured by using the method of Example 1-2 and curcumin (Curcumin@MB) and paclitaxel (PTX@MB) are used as the model drugs to manufacture a suspension-formed microbubble drug delivery system. 1.5 ml of the suspension is each divided into plastic pasteur pipettes and each plastic pasteur pipette containing the suspension is vertically fixed to a stand. Then, focused ultrasound of 5 MHz is irradiated for 1 minute according to power (1 W, 5 W, 10 W, 15 W) and concentration of released drugs is analyzed. The condition of ultrasonic waves is focused ultrasound having the frequency of 5 MHz. The result obtained by identifying curcumin is shown in FIG. 4A and the result obtained by identifying paclitaxel is shown in FIG. 4B.

As identified in FIGS. 4A and 4B, when ultrasonic waves is irradiated, drug releases in curcumin and paclitaxel are all increased according to irradiated ultrasonic power (1 W, 5 W, 10 W, 15 W), compared with those in control groups to which ultrasonic waves is not irradiated. Accordingly, it may be identified that the drugs carried in the microbubble including the first and second surfactants may be efficiently and quickly released by focused ultrasound, which is external stimulus.

### Example 4: In-Vitro Cell Experiment of Manufactured Microbubble Drug Delivery System

### Example 4-1: Cytotoxic Experiment of Manufactured Microbubble Drug Delivery System

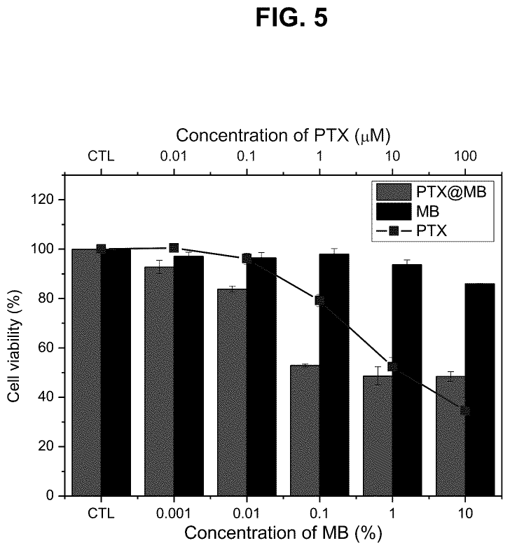

An experiment is performed to identify cytotoxicity of the manufactured microbubble drug delivery system and the drug delivery system loading the drugs. The experiment is performed after an experimental group manufactured by using the method in Example 1-2 (microbubble drug delivery system loading paclitaxel PTX@MB) and a control group (microbubble without loaded drugs MB) are deaerated to burst bubbles. Each solution is added to U87MG cells to respectively set final concentrations to 10%, 1%, 0.1%, 0.01%, and 0.001% and final concentrations of free paclitaxel are respectively set to 100 μM, 10 μM, 1 μM, 0.1 μM, and 0.01 μM. 5000 cells are grown in each well and cells are cultured for 72 hours. Then, a solution containing a cell culture fluid and tetrazolium salt derivatives are added to measure cell viability and in-vitro cytotoxicity of each material is identified. The results are shown in FIG. 5.

As identified in FIG. 5, in case of free paclitaxel, cytotoxicity is hardly measured at low concentration, however, may be identified at concentration of above 1 μM. It is identified that cytotoxicity is not indicated in the microbubble without loaded drugs which is the deaerated control group MB even to concentration of 10%. In case of the experimental group PTX@MB, cytotoxicity to a cell line, which is glioblastoma, may be identified from concentration of 0.1%. In this regard, it may be identified that since cytotoxicity does not exist in the control group, cytotoxicity existing in the experimental group is due to the model drug (Paclitaxel) carried in the manufactured microbubble drug delivery system. Accordingly, the manufactured microbubble drug delivery system shows high drug loading amount and thereby, may be efficiently used as a drug delivery system.

### Example 4-2: Analysis on Absorption Degree within Cell in the Manufactured Microbubble Drug Delivery System

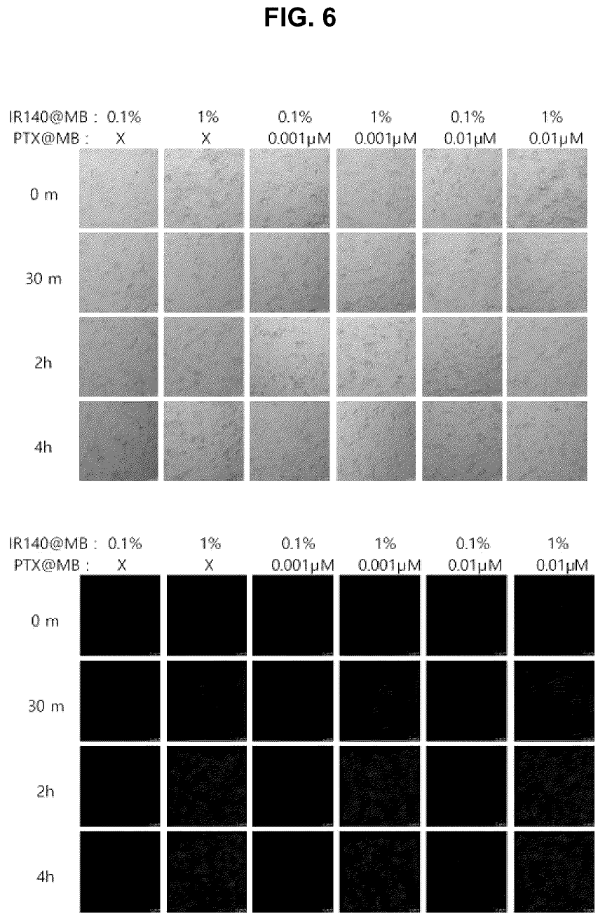

An absorption degree of the microbubble drug delivery system loading the model drug (Paclitaxel) and the microbubble loading the fluorescent dye (IR140) within cells is analyzed according to their concentrations. The experiment is performed after each microbubble is deaerated and burst. The final concentration of the deaerated microbubble drug delivery system PTX@MB loading the model drug (Paclitaxel) is set to 0.01 μM, 0.001 μM, and 0 μM to be used and the final concentration of the deaerated microbubble IR140@MB loading the fluorescent dye (IR140) is set to 1% and 0.1% to be used. Both solutions are added together to U87MG cells, a cell culture fluid is renewed before observation to remove materials that are not absorbed to cells, and then, observation is performed by using a confocal microscope. The results are shown in FIG. 6.

As indicated in FIG. 6, regardless of the concentration of the model drug (Paclitaxel), an absorption amount of the deaerated microbubble loading 1% of the fluorescent dye (IR140) within cells is high.

According to the present invention, the microbubble including the first surfactant, which includes a polyethylene glycol (PEG) based nonionic surfactant, and the second surfactant, which includes a Tween-type surfactant or a SPAN-type surfactant, may stably have uniform size distribution, may include high loading amount of drugs and dye on the surface of the delivery system, and may not have a drug agglomerate. Accordingly, the microbubble may be efficiently used as a drug delivery system.

In addition, according to the present invention, drug release may increase by ultrasonic wave, which is external stimulus, from the microbubble and thereby, more efficient drug delivery may be available.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of manufacturing an ultrasound-induced microbubble drug delivery system comprising:
(a) manufacturing a mixture by dissolving drugs, dye, or a combination thereof; a first surfactant comprising a polyethylene glycol (PEG) based nonionic surfactant; and a second surfactant comprising a Tween-type surfactant or a SPAN-type surfactant, in an organic solvent;
(b) manufacturing a concentrate by removing an organic solvent from the mixture; and
(c) manufacturing microbubble using a sonicator after putting liquid gas into the manufactured concentrate.

2. The method of claim 1, wherein the microbubble has a size of 0.5 μm through 30 μm.

3. The method of claim 1, wherein the drug delivery system is dispersed in an emulsion-form aqueous solution.

4. The method of claim 1, wherein the organic solvent comprises dichloromethane, ethyl acetate, acetone, ethanol, methanol, methyl ethyl ketone, methylene chloride, dichloroethane, chloroform, dioxane, dimethyl sulfoxide, acetonitrile, acetic acid, or combinations thereof.

5. The method of claim 1, wherein the liquid gas comprises Perfluoro-n-pentane, 1H-Undecafluoropentane, or 2H,3H-perfluoropentane.

6. The method of claim 1, wherein the polyethylene glycol (PEG) based nonionic surfactant is selected from the group consisting of Pluronic F-127, polyethylene oxide-polypropylene oxide-polyethylene oxide (PEO-PPO-PEO), polystyrene-co-maleic anhydride, polyethylene glycol-poly L-lactic acid-polyethylene glycol (PEG-PLLA-PEG), a copolymer of polyethylene oxide and polypropylene oxide, and combinations thereof.

7. The method of claim 1, wherein the first surfactant and the second surfactant are included in weight ratio of 1:0 through 1:1.

8. An ultrasound-induced microbubble drug delivery system manufactured by using the method of claim 1.

9. The drug delivery system of claim 8, wherein drug release is increased by ultrasonic irradiation.

10. The drug delivery system of claim 8, wherein the system comprises fat soluble drugs.

11. A contrast medium comprising the ultrasound-induced microbubble drug delivery system manufactured by using the method of claim 1.
